(19) United States
(12) Patent Application Publication
(10) Pub. No.: US 2019/0088373 A1
(43) Pub. Date: Mar. 21, 2019

(54) Automated Assistant For Remote Patient Tracking, Diagnosing, Alerting, And Prevention Of Heart Diseases, Cardio Warning Service/System

(71) Applicant: Alberto Sarmentero, Arlington, VA (US)

(72) Inventor: Alberto Sarmentero, Arlington, VA (US)

(21) Appl. No.: 16/133,182

(22) Filed: Sep. 17, 2018

Related U.S. Application Data
(60) Provisional application No. 62/559,300, filed on Sep. 15, 2017.

(51) Int. Cl. G16H 80/00; G16H 10/65; G16H 15/00; G16H 40/20; G16H 40/40

(52) U.S. Cl. CPC G16H 80/00; G16H 10/65; G16H 40/40; G16H 40/20; G16H 15/00

(57) ABSTRACT

An automated remote service and systems are illustrated with their associated devices that includes wearable sensors and mobile apps for autonomous patient monitoring, diagnosis and alert device and its related software including inference rules, Artificial Intelligence, and Big Data algorithms and techniques for the prevention of diseases.

### CROSS-REFERENCE TO RELATED APPLICATIONS

This application claims priority from U.S. Provisional Patent Application 62/559,300, filed Sep. 15, 2017, the contents of which are hereby incorporated herein.

### BACKGROUND

Field of the Invention: Digital Health, Healthcare software, Healthcare devices, Digital Therapeutics, Healthcare-related Artificial Intelligence and Machine learning.

The invention pertains to the field of digital health, healthcare software and hardware designed to detect, alert and prevent heart diseases and some other diseases or abnormalities in the human health conditions by using Telemedicine and blockchain technologies.

More particularly, the invention pertains to the field of Healthcare-related Artificial Intelligence, Machine learning, Big Data analysis, Internet of Things, blockchain and wearable sensors combined and applied to remotely track the main vitals and warning in real time if/when any abnormality in the health state arises.

### BRIEF SUMMARY

Around 33% of the causes of death of human beings nowadays worldwide are due to cardiovascular diseases, according to World Health Organization (WHO) and other international health agencies. Early signs of heart disorders are hard to interpret correctly without a very specialized medical education. This invention of Cardio Warning has been designed to help resolving this issue with an up-to-date algorithm based on deep AI+Big data model which can identify and alert Cardio Vascular Diseases (CVD) and/or further other health problems in real time.

Additionally, according to U.N. forecasts, in 2045 the number of persons in the Old age is expected to be larger than children worldwide. In the meantime, the developing countries will have to achieve better results with less resources, including Health Care Services, leading them to implement a preventive Health Care System instead of a reactive one in order to reduce Health Care costs.

Cardiovascular diseases, cancer, and diabetes are the main mortality causes in the world (70%) specially when ages of the top of the population pyramid are increasing every year (>45 years→6.8%; >65 years→6%-10%; >85 years→15%-20%). As said above, one third of human deceases worldwide are related to heart dysfunctions but it further implies that the 70% of the overall hospitalization costs (around ⅔) are caused by heart pathologies.

Telemedicine and technological advances play an important role in streamlining costs rationalization by getting prepared for early detection of heart diseases and continuous patient tracking, while improving the capability of the specialists to attend a bigger number of patients.

Half of the people who die because of cardio-vascular diseases (CVDs) could be alive if they were diagnosed and alerted during their early symptoms, which not always is feasible today. This Cardio Warning invention is aimed to allow CVDs to be remotely and constantly diagnosed by cardiologists, as long as the Artificial Intelligence (AI) models of Cardio Warning are able to identify CVD problems by tracking the first symptoms of them through signals from a wearable platform of small and smart biometric sensors.

Official Healthcare organizations worldwide are constantly seeking formulas to guarantee the future financing of their Healthcare systems by reducing the present-day dramatic increasing expenditure of the sanitary services provided. The most promising way to help them accomplish this target relays in improving the research processes for the development of new cost-effective products and services.

The combined costs of medical prevention, diagnosis and treatments have reached a growing rate as much as twice the one of GDP worldwide. The increase in life expectancy of many users of healthcare organizations and therefore the treatments due to declining-health persons will increase the expenses year after year in the developed societies.

On the other hand, in many underdeveloped health system countries, the very high rates of population growth and the cost of treatments versus the low income rate per family prevents many citizens from accessing even any basic healthcare.

Currently, due to the costs involved and the knowledge required for early heart illness detection, these diseases are hardly noticed by the primary health services leading to a situation where only 30% of the cases are detected [European Heart Journal (1999) 20, 421-428] and even less correctly treated.

Heart diseases are not just difficult to detect but expensive to treat as the usual treatment includes visits to the specialist and often hospitalization when the disease is in an advanced stage. Only treatment of Congestive Heart Failure (CHF) represents 2% of the U.S.'s GDP according to Oxford Journal (and similar rates apply in Europe) that affects between a 2% and 3% of the total world population. Precisely hospitalization represents 60% of the total cost of the treatment in developed countries according to the European Society of Cardiology [European Journal of Heart Failure 2001] (see FIG. 3).

The sooner a disease is treated, the less surgical intervention required and at a lower cost as a continuous and active adjustment of the treatment in its early phases reduces the necessity of hospitalization [MIT Technology Review (2011)].

The dispersion of medical information from the patients often renders ineffective the management of their data and therefore affects the value chain that can be applied to them.

On top of this, there is an exponential increase in the complexity of the system that has led to serious fragmentation of such medical data, causing healthcare organizations to frequently lose their focus on the patient.

Dispersed data results in ineffective information, so multiplying the cost of acquiring or developing knowledge about health prevention measures while making it difficult to access models that benefit medical research.

During the last years, human lifespan expectancy has increased enormously and nevertheless the quality of life in their older years results very damaged by different ailments and illnesses. Specifically, cardiovascular diseases (CVDs) are the leading cause of death in the world today, causing more than 17 million reported human deaths annually while many more millions of people remains usually with a low quality of life. All the signs indicate that the only way to fight and reduce these ailments is through prevention.

However, due to the aging of the population and the saturation—even the collapse—of many health services around the world, on the one hand, and the inertia of many health organizations to address this problem, Preventive measures available to fight CVDs are generally minimal or nonexistent, causing a high rate of treatments, hospitalization, medications, surgical interventions, etc.

Periodic or continuous monitoring of the heart allows an early diagnosis and therefore the application of initial phase solutions that would avoid and/or delay most heart diseases, saving many lives and improving the quality of life of people, in addition to reducing drastically the costs required.

Similarly, the knowledge and necessary means to perform—and especially to analyze electrocardiograms (ECG) for proper diagnose—are not accessible to large areas of the World (small health centers, rural areas, places with difficult access, less developed countries, etc.), still making more difficult the early detection of heart diseases.

Although CVDs are not any infectious agents, in recent years and given the percentage of people affected throughout the planet, cardiovascular diseases have reached the level of pandemic. At present, 30% of the deaths that occur in the world (45% of those in Europe) are due to cardiovascular problems, but the most worrying fact is that only 30% of them are detected in time. Some of the key factors behind these numbers are:

Cardiovascular diseases usually find difficult conditions to get properly diagnosed in many primary care centers or even hospitals, although they often should show there the first symptoms. The main reason is that when people goes to a hospital, the symptoms may not appear at the time of performing the electrocardiogram.

70% of heart diseases are not diagnosed until the problem causes damage or it is already difficult to solve. This is mainly due to the fact that very often there are no preventive methods used.

This situation, together with the aging of the population, results in a very high endowment of resources to alleviate the consequences of these ailments, generating a high social and economic cost for the majority of affected countries.

The WHO (World Health Org) already indicates that 80% of myocardial infarctions and strokes are preventable. The key is (as in most diseases) a periodic monitoring that allows an early diagnosis and treatment in the initial phases. Therefore, preventive technologies like this invention would greatly reduce these economic, social and labor costs; and most importantly, they would save countless lives, improving the quality of life of many others.

The current solutions mainly go only through reactive strategies that—in many cases—have associated costly, palliative or chronic treatments that imply a huge economic effort for the different health systems, aggravated by the increase in longevity while maintaining serious imbalances and unhealthy lifestyles.

These reactive strategies are unsustainable in the short term. The solution implies both coordinated preventive strategies and preventive technologies, which are still very incipient. Currently the main way to detect any CVD is based on the monitoring of the heart through an ECG of those patients with some symptom. However, in many cases, there are not enough specialists in Cardiology available and/or equipment to properly monitor or diagnose the ECG; or simply the symptoms do not appear at the time when the ECG is performed.

In high-risk cases, the patient is monitored for 24 hours, either directly in the hospital or through a device (holter) that monitors and registers the patient's ECG during that time so that a doctor can analyze it later. In many third world countries, the problem is aggravated because of the lack of monitoring, as they use to have specialized MD's only in the main hospitals. In the case of some medical insurers for companies, they already include periodic stress tests and ECG for employees with some risk factors since with that prevention they save a large amount of money. However, these tests imply that the patient must go to these clinics.

Although it seems that everyone agrees that prevention is the best way to save lives and also medical costs, the several large insurers and medical companies are barely taking the first steps in this direction and in general the health care organizations are not yet all making the necessary fast steps to adapt themselves to new preventive technologies, by far much more “health-effective and cost-efficient.”

To begin to apply such preventive strategies on these diseases it would be necessary the appearance in the market of simple and inexpensive devices that allow to monitor the ECG and other constants of the patient, and have automatic diagnostic systems. This would be the right way to detect the first symptoms of these ailments at an early stage.

In order to prevent heart disease and reduce hospitalizations once the disease is developed new systems allowing early detection and diagnostic as well as remote patient tracking are needed, and here is where Cardio Warning System and Service of this invention plays a fundamental role.

There are already some wearable devices in the market and some others in developing process. They have no ability to accurately analyze electrocardiograms. Cardio Warning main competitive advantage comes from its analysis software.

There are also some research projects in course on this matter but they have mostly been designed for hospital use (not for personal outdoors use). These are developments to aid diagnosis but they are not expected to operate autonomously, nor to reaching the same percentages of success in the analysis of electrocardiograms.

Half of the people who die because of Cardio-vascular diseases could be alive if they were diagnosed and alerted during their early symptoms. A CVD can be early diagnosed remotely by cardiologists with Cardio-Warning, a system/service of this invention that includes an AI model able to identify CVD's problems by tracking the first symptoms of them through a platform of sensors.

Cardio Warning also works as an assistance system that includes portable biomedical sensors and a service for preventive diagnosis through remote monitoring of patients with heart disease to anticipate the evolution of this disease through rules of inference and Machine Learning Analysis techniques. ML analytics with the aim of reducing the number of hospitalizations and improving the quality of life of patients.

### BRIEF DESCRIPTION OF THE DRAWING FIGURES

Exemplary embodiments will hereinafter be described in conjunction with the following drawing figures, wherein like numerals denote like elements, and:

FIGS. 1A-1B illustrate the evolution of population age pyramid according to Oxford Institute of Ageing. For year 2045 the number of persons in the 3rd age is expected to be bigger than children worldwide;

FIG. 2 represents a cost distribution of the treatment in developed countries according to the European Society of Cardiology, where 60% of the costs come from hospitalization. Cardio Warning target is to reduce hospitalizations thanks to early detection and preventive treatment;

FIG. 3 displays an estimation of cost intensity for the treatment depending on intervention phase according to the Disease Management Association of America; the sooner the disease is identified and behaviors are changed, the less the treatment cost;

FIG. 4 shows the size of current devices used for health monitoring. Device showed in FIG. 4 is Cardio-MEMS;

FIG. 5 shows the complete Cardio Warning cycle, from retrieving patient data from wearables and/or implants to the final report to health care professionals or alert systems, passing through the transmission to a central repository for their analysis;

FIG. 6 shows the high-level software architecture for Cardio Warning Service;

FIG. 7 shows one portion of the classification algorithm developed for cardiac diseases, in particular for atrioventricular block arrhythmias;

FIG. 8 shows the example of algorithm for the specific case of a third-degree AV block; and

FIGS. 9A-9C show one portion of how the classification and diagnose model is translated to specs used to develop and code the Classification and Diagnosis Modules (CDMs); in this case just one part of the ECGs module for arrhythmias;

FIG. 10 illustrates example interest data in accordance with one embodiment.

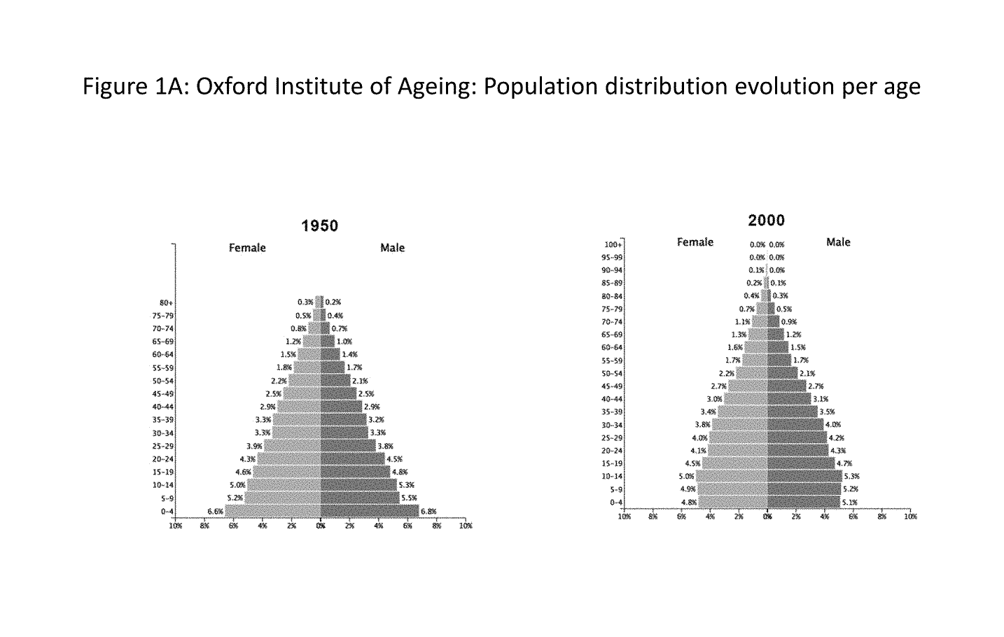

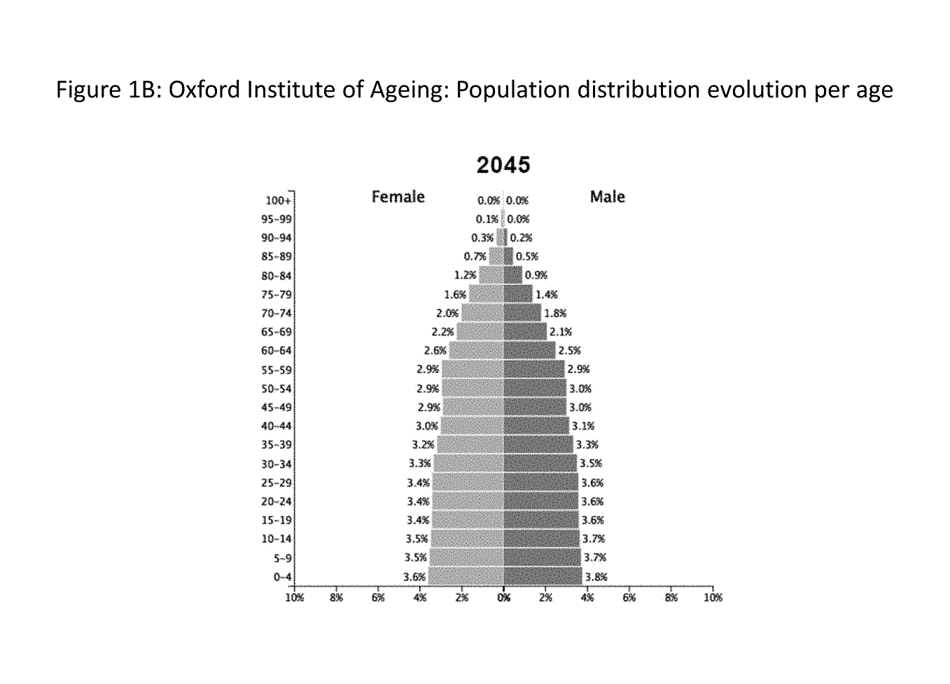

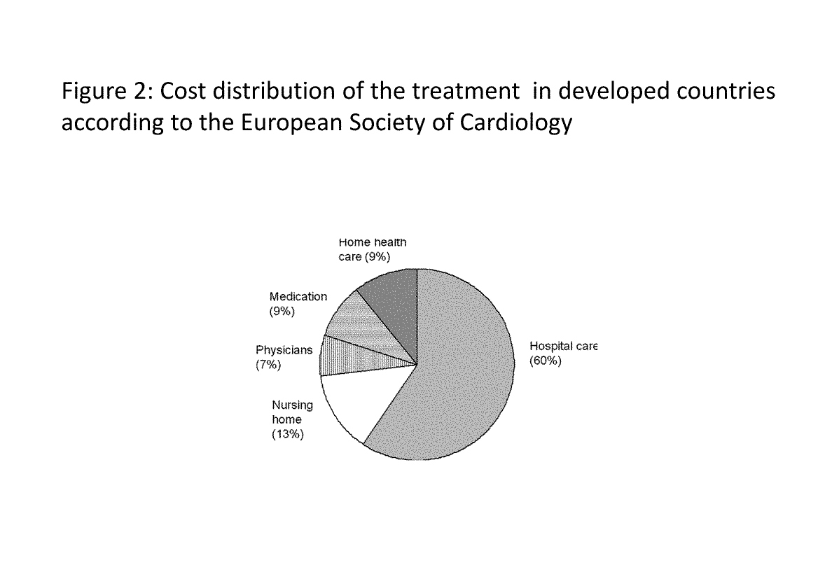

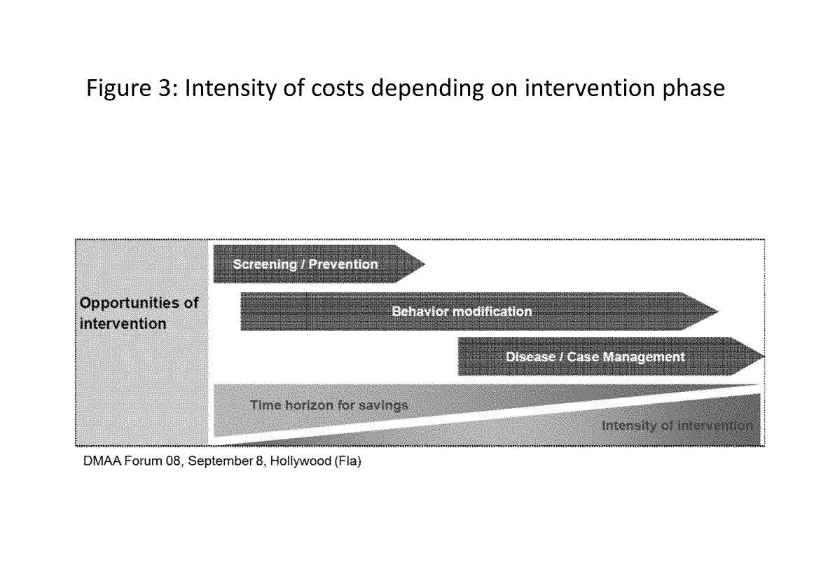

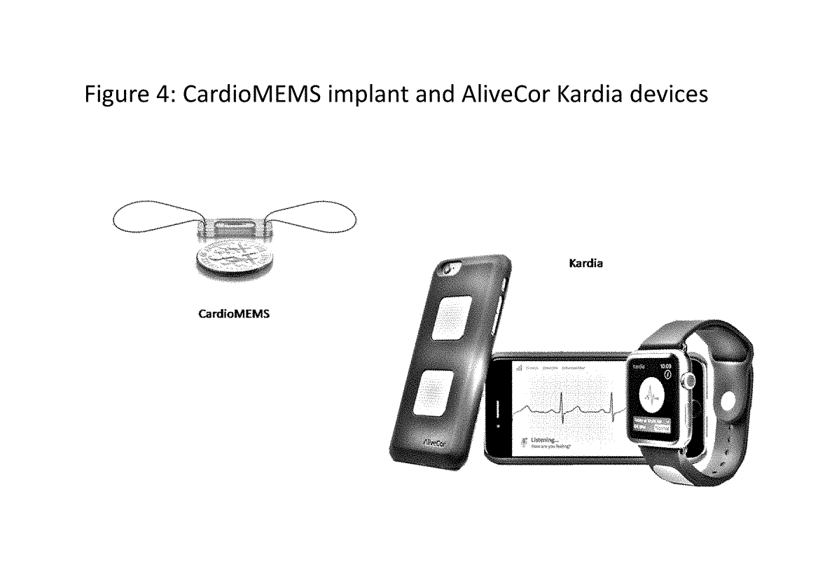

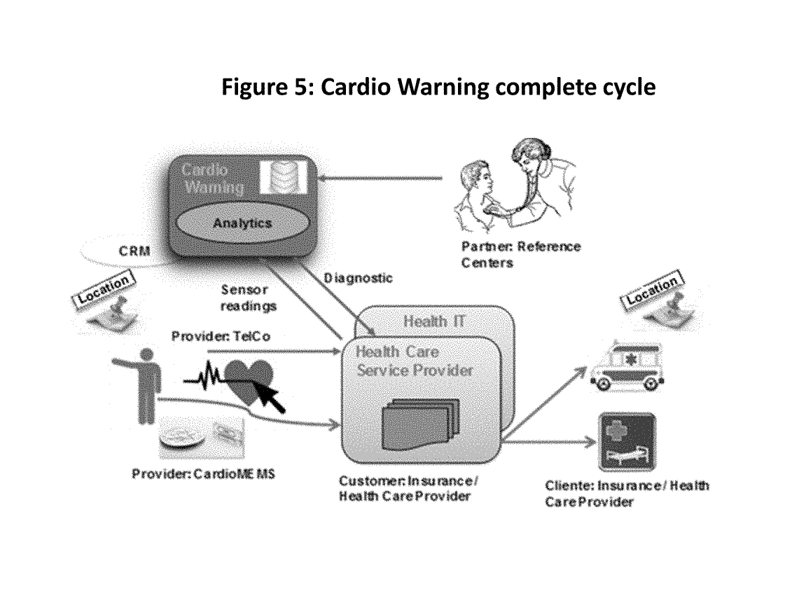

### DETAILED DESCRIPTION

Cardio Warning System and Service (CW) is a Remote Diagnosis Aid Service for heart diseases through remote patient monitoring, anticipating the disease evolution through inference rules and massive data analytics techniques.

The main objective of this invention is to improve healthy life expectancy for patients with heart and/or heart related diseases and reduce the number of hospitalizations, and so reducing Health Care costs while improving the autonomy of the patients.

Cardio Warning System (CWS) represents a device for end users with the possibility of linking it wirelessly with any online or delayed medical service. The invention includes a unique algorithm, which notices any abnormalities in heart's operation and provides recommendations to take preventative measures. AI technology can so predict heart diseases, using the specific model developed with the collaboration of top professional cardiologists and cardio-vascular surgeons.

Nowadays the development of wearables retrieving vital signals is increasing every year (i.e.: CardioMEMS, approved by FDA in 2016, or AliveCor Kardia, which converts any smartphone (Android, iPhone, etc.) in a portable electrocardiographic device using mobile devices in many cases to store the information.

CardioWarning (CW) consists of a wearable Biomedical System and Service based on Artificial Intelligence and Big Data analysis applied to Cardiologic and other medical data. It is a solution for continuous assisting in the diagnosis of cardiovascular and other diseases. Its software has been designed as a neural network ready to interpret electro-cardiogram signals and immediately Warning of any possible arrhythmia or heart anomaly directly to the smartphone of the user and to user's MDs or local emergency services—through IoT—from any point of the planet.

In association with its wearable sensors, CW performs a continuous monitoring of the heart with the main associated parameters to give a total coverage in the early prevention of heart disease, in connection with online medical services or on demand and Real-time alerts, Big data technology for pattern recognition and patient tracking, while staying on the ground of preventive healthcare by thwarting occurrence of CVD and/or other diseases.

The most important component of this invention is this biomedical software combined with a variety of hardware apparatus, all this designed to detect, alert and prevent heart diseases and some other diseases by using specific sensors included in wearable items for specific data gathering with wide functionality and its compilation by remote sensing and its transmission to the Cloud storage by several alternative ways/items. Several-level analysis applying Big data with Artificial Intelligence results in quick response to any anomalies in the health state by sending messages by Internet of Things (IoT) when determined alarm signals arises.

The core of Cardio Warning is its biomedical software development based on artificial intelligence, big data, and analytics algorithms which processes medical data received from sensors located at patients with risk of heart diseases.

Cardio Warning retrieves the information from the mobile device wore by patients using any of the suitable current technologies (Bluetooth, Wi-Fi, etc.), to transmit the information to a central server using available Cloud services (cloud storage, location, data analysis).

All the information received is stored in a Cardio Knowledge Database generated with the support of the centers of reference in Cardiology and is processed by means of inference procedures looking for patterns already defined by these hospitals to early detect any sign of illness and at the same time provide results to local health professionals who assist, diagnose and remotely monitor patients in real time.

In the case that any anomaly is detected, the Health Care professionals contact the patient to prescribe a medical treatment preventing the evolution of the disease or even in the most urgent cases requiring immediate action Cardio Warning raises an alert if it is necessary for immediately sending an ambulance or a specialist to the patient's location.

Cardio Warning is fundamentally oriented to health service providers (private and public providers), hospital networks, Health-IT companies, or even individual customers, as the service is oriented to diagnose any pathology susceptible to be diagnosed following medical procedures and rules from data received remotely from any device using our interface.

Cardio Warning System is Associated with our own area defibrillator locator application for smartphones. Included in this invention, this APP displays a network for a quick location of the closest defibrillator available when necessary and wherever the user is located in the world.

A number of different sensors available in the market can be added to the Cardio Warning wearable instrument of this invention. The wearable sensor-holder platform uses electrodes and other non-invasive electronic technology to perform biometric readings. They can be able to measure the most important body parameters (Oxygen in blood, ECG (EKG), glucose, respiratory rate, airflow, Skin temperature, pulse, Heart rate, Blood pressure and fall detection). All this biometric data is encrypted and sent to the Cloud storage following strict transmission protocols and security requirements in real-time to be visualized online on the MD and/or the patient's private account.

Cardio Warning software is based on a cloud service receiving data from different sources (personal devices, hospital systems, web connections, etc.); these data (ECGs, glucose, blood pressure, etc.) is processed to obtain a diagnose and further start the medical procedure/s associated to the disease detected. The software is composed of the following components:

Client App (CA): a client designed to be installed on mobile devices in order to facilitate the integration with the Cardio Warning service to existing and/or new apps.

Incoming Interface (II): an interface in charge of receiving all data from external sources relevant for the diagnosis (i.e.: ECGs, blood pressure, glucose, breath information and rhythm, patient position and orientation, geo-localization, biodata, etc.). Medical standards are used; however the interface can be expanded to accept any specific requirements of existing devices.

Data Storage Instances (DSIs): for each user, a new instance is created for storing data received (continuously, periodically, or in a single burst) from the user session. In case of continuous data flow Internet of Things software modules are employed to continuously feed the user personal database with his/her data (refer to point [0067] for additional information).

Customer Historical Data Storage (CHDS): data is stored in cloud databases with all the security guaranties to secure customers privacy. Each type of data retrieved from customers is stored in separate tables to look for diagnosis in just one type of disease or look for crossed relationships between different symptoms (please refer to point [0077] for additional information).

Data Analysis Modules (DAMs): there is a different DAM module per type of disease to be analyzed. DAMs modules extract the relevant information from raw data stored in the CHDS and prepare it to be analyzed by the Classification and Diagnosis Modules (CDMs).

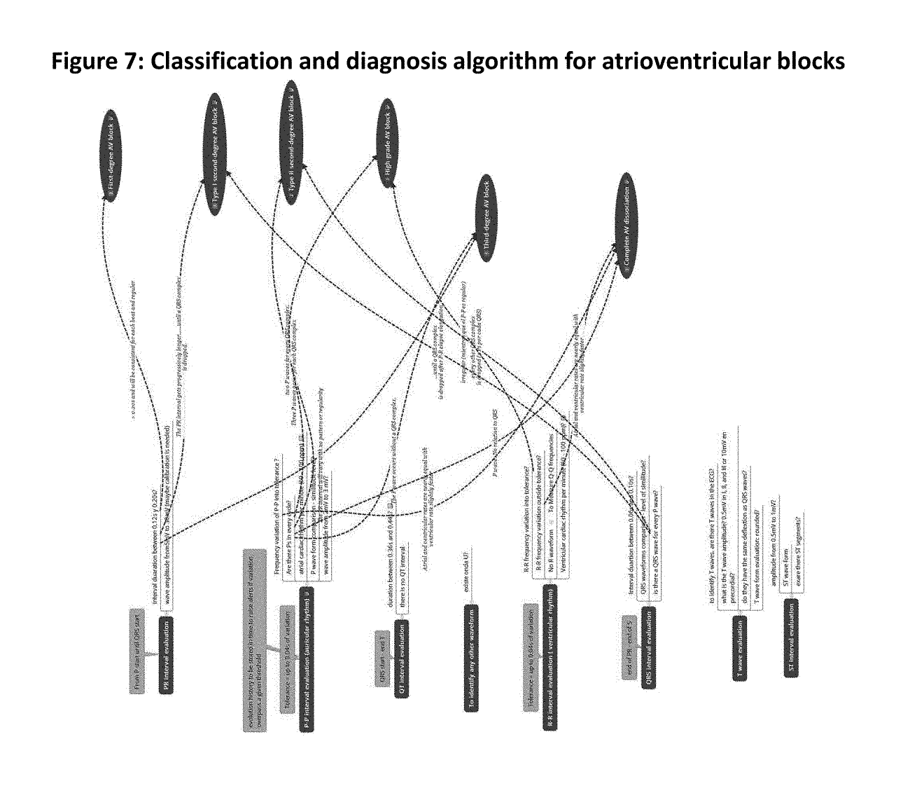

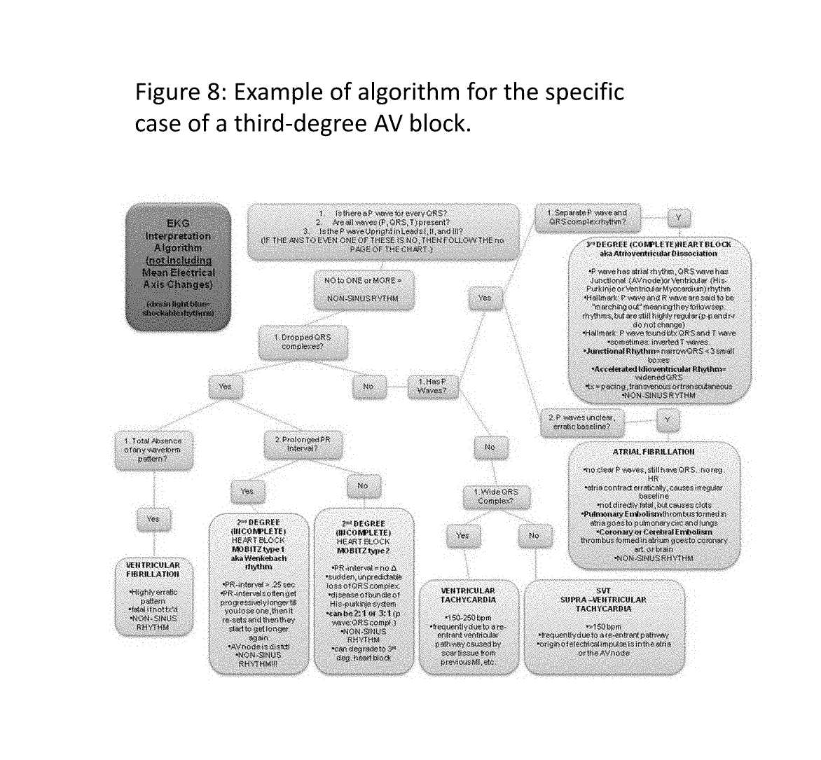

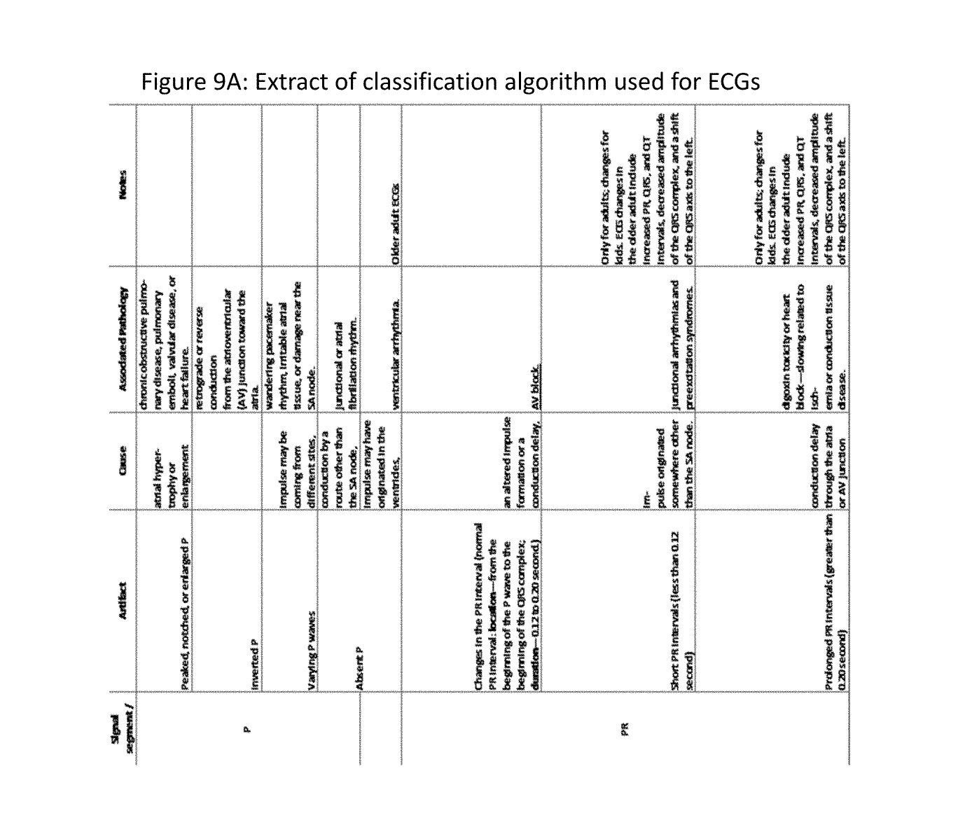

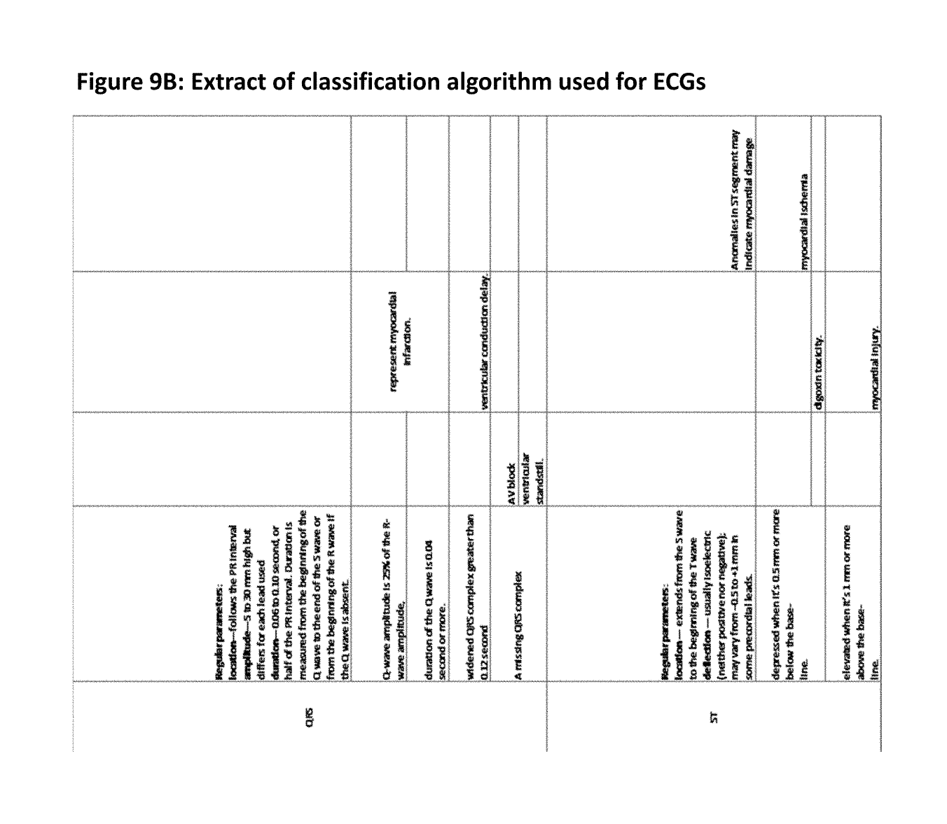

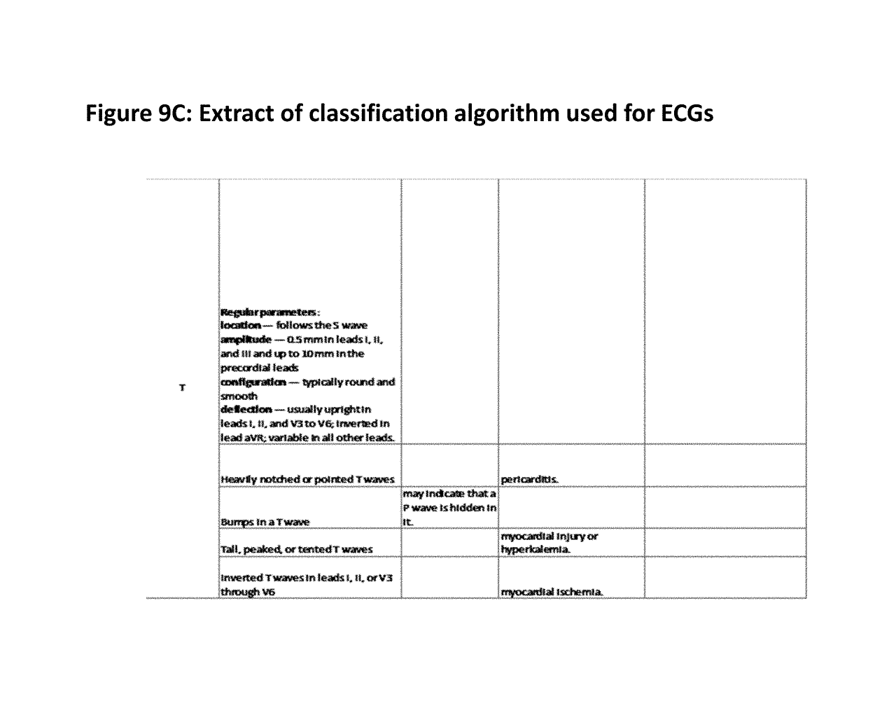

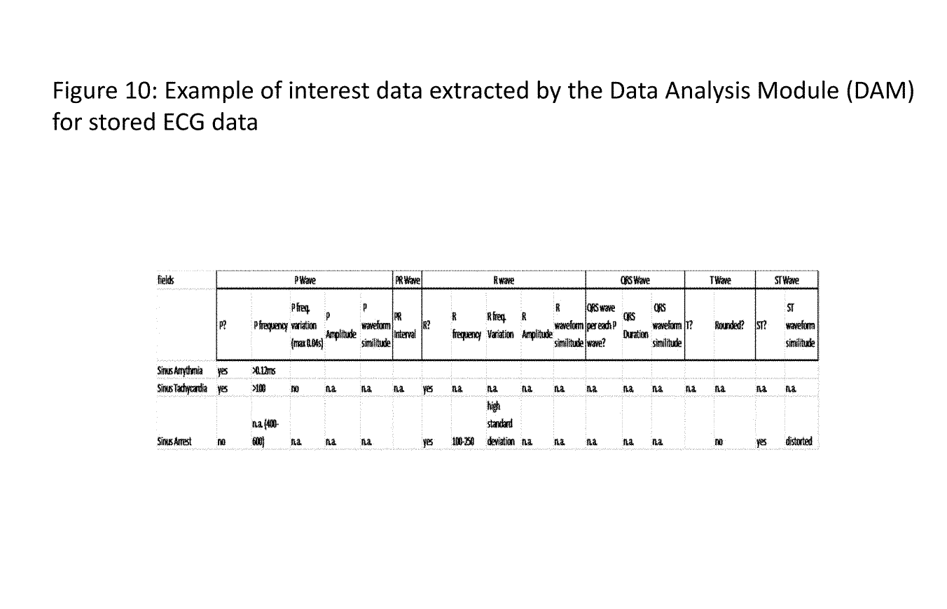

Classification and Diagnosis Modules (CDMs): there is at least one CDM instance per type of disease and customer running concurrently. CDMs run continuously analyzing data extracted by the DAMs identifying any anomaly or artifact in the data received from each customer using multiclass classification algorithms, neural networks, and Big Data technics to perform the most accurate diagnosis on each patient. Referring to FIGS. 7 and 8, Diagnose modules are continuously upgraded with the results obtained from the Disease Data-warehouse and Inference Component (DDWH & IC) (refer to point [0077] below for additional information) in order to continuously improve diagnose capabilities.

CDMs are executed depending on the periodicity of data received per customer. In those cases where a periodic or a continuous data feeding is performed CDMs are scheduled in a real-time manner (i.e.: for detecting a heart attack). If the service is used in the “one-shot” modality (under demand); only one instance is created to attend the demand on the data associated (received as part of the service demand)

In accordance with another embodiment, the system includes an AI system configured to engage in an interactive conversation with a patient. In this way, the AI system administers a survey, which then serves as the input for another neural network system. Thus, the AI module acts as the agent conducts the survey.

If anomalies or artifacts corresponding to a potential disease are detected on customer's data, a notification is sent through EICMs (refer to the following paragraph for additional information) for alerting the customer, emergency services, health care institutions, insurance, relatives, and any other services subscribed to Cardio Warning service.

External Interfaces and Communication modules (EICMs): this module allows the customer to configure how notifications and alerts are delivered and to whom. There is one module for each company, institution, service, or platform subscribed to the diagnose results obtained using Cardio Warning service.

By default, any notification is sent directly to the customer, and is the customer who decides who other third-party providers receive notifications and alerts (and the types of alert to be sent to each third-party).

Close Help Display Component (CHDC): If a critical situation is diagnosed, as part of the mobile client, a map with the devices that can provide immediate help, is shown on the patient (and relatives and third-party devices described in the point [0073] above) mobile phone/s showing where the device/s is/are and their usability status.

This component also includes the main tools to create, update, and maintain the database with the help-devices available (i.e.: defibrillators) for each area, allowing to identify “Health-Protected Areas” on each city in the world.

Disease Datawarehouse and Inference Component (DDWH & IC): All pathology data received is stored and organized in a Disease Data warehouse (DDWH) without any reference to patient precedence. Over these data, inference algorithms are executed continuously in order to detect insights on the diseases improving diagnosis rules used at Classification and Diagnosis Modules (CDMs) or even establishing symptoms and relationships not currently known for improving earlier detection and prevention.

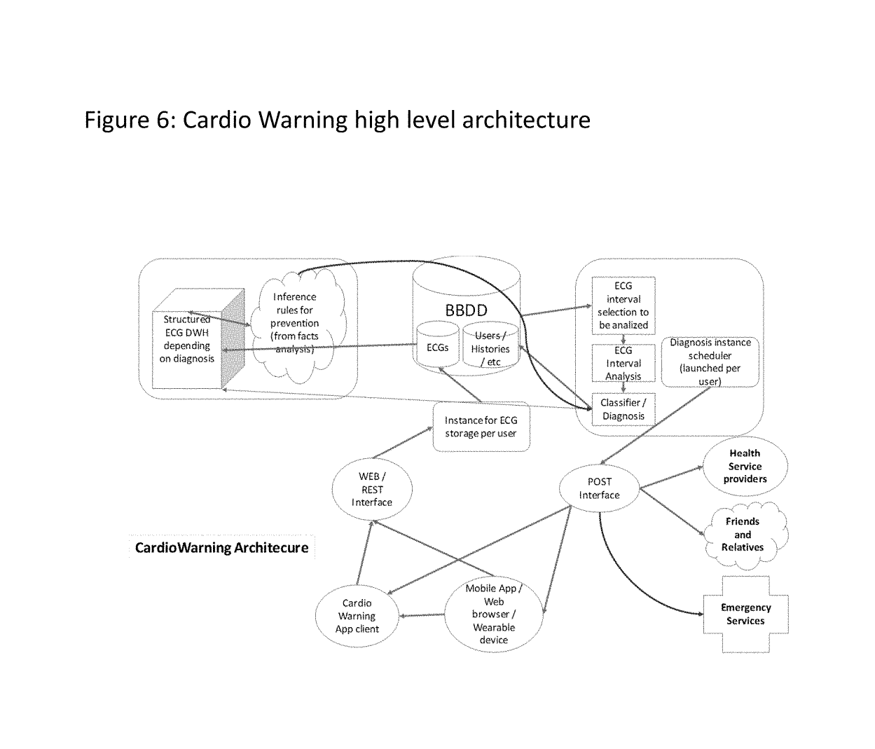

High level modules and relationships between then can be seen in FIG. 6. Among the main said clinical functions measured by the wearable platform of sensors—in a patch, special shirt or sensor's vest—are the electrocardiograms (ECG) in multiple-lead configuration that allows the ability to pre-diagnose symptoms related to cardiac malfunction, such as detection of arrhythmias, Pre-infarction, angina pectoris, among others. It includes measures of body temperature through an infrared sensor Thermometer with an accuracy of 0.03° C.

Also Reflective Pulse Oximeter (RPO), with capacity to determine the oxygen content in the blood and the cardiac pulse, Impedance Pneu-monography: determines the respiratory rate measured in ventilations per minute. It helps to diagnose apnea, Chronic Obstructive Pulmonary Disease (COPD), and other pathologies and all these include programmed alarms when readings arise over or under certain pre-established parameters.

Diagnosis of biomedical signals and their comparative analysis to perform such early alarms, especially the ones related to anomalous cardiac conditions by means of the integrated system of this invention that allows to make a comparative recognition of biomedical patterns in real time. This system allows to generate early alarms of parameters of cardio, oxygen in blood, body temperature, mobility, as well as autonomous detection of respiratory rate and apnea detection, among other important parameters.

Remote auscultation can be also performed by means of the embodiment of an auscultation device. Such embodiments can employ, at least in part, one or more of several commercially available wireless receiver devices, such as, without limitation: headsets and headphones, mobile phones and/or other computer devices and/or electronic devices.

Another sensors used in the context of this invention include the fall detection with a real-time biometric multi-parameter sensor using a Smartphone App mainly for the use of people with chronic diseases related to cardio, obstructive pulmonary hypertension and other diseases. This also include a 3-axis Accelerometer that detects body mobility, verticality, permanent use pedometer that measures how far someone has walked by counting the number of times the feet are raised and put down again for the control of obesity, detection of fall, insensibility and unconsciousness.

The Cloud Storage, processing and retrieval service of this invention includes intelligent (AI) detection and notification of various medical anomalies and can recognize and alert on several diseases and medical conditions including heart attack and stroke. In addition, this information stored in the cloud can be permanently monitored or viewed in real time by sending its processed data directly to the smartphone of the users and/or their Medical Center.

Cardio Warning provides due assistance to the specialized diagnosis of first level regardless of the location of the patient, anticipating the clinical episodes, reducing response times, treatment and hospitalization of patients with heart disease. Detecting an arrhythmia on time is key to preventing serious episodes such as a stroke.

Cardio Warning algorithms based on Artificial Intelligence do ensure automatic real-time monitoring in unmatched detail. The sensors automatically send all the data collected to an App/Service designed to engage patients to self-empower by monitoring and help improving their overall health.

The Cloud-based healthcare management service allows access to visualize clear and objective patient information in real-time, and deliver higher quality of care with more personalized medicine, earlier intervention and better prevention. The solution consists of 5 modules that work as a complete solution for the monitoring and diagnosis of heart disease:

A system of analysis and diagnosis of the ECG that allows to correctly obtain all the parameters of the electrocardiogram, and perform an analysis of the patterns to diagnose the possible heart diseases of the patient.

An inbound/outbound integration module that allows both to load in the system any type of ECG for further analysis and diagnosis, and to allow access to patient data to the different associated medical services. This module facilitates access to the service to any person/institution/hardware that needs to perform a diagnosis of an ECG, and allows medical professionals to access the results/ECGs of their patients.

An access point (App, Website) through which it can be received the ECGs from our own hardware devices or the ones from other manufacturers, as well as those ECGs to be directly added by the patients/medical centers. The result will always be the interpretation and possible diagnosis of this ECG. A hardware device with sensors that allows patient monitoring of both the ECG and other parameters to expand the diagnosis, such as respiration, temperature and accelerometer.

This device includes both with sensors for reading on demand, and in continuous monitoring format (patch, shirt, etc.). The device is directly connected to the access point to obtain an immediate diagnosis and to receive the corresponding alerts, as well as an emergency alert system for a serious cardiac situation.

A Big Data system that monitors the evolution of ECG & apps, and is responsible for finding new unknown patterns that evolve to heart disease. This system is the key to an even more premature detection of any possible cardiovascular problems and a future helping tool in the general medical and specific cardiological research.

A Permit Management System by which the patient can give access to the desired doctors/services, as well as sharing his medical data anonymously with researchers and pharmacists even in exchange for a reward if so agreed.

Cardio Warning is a solution for assisting in the diagnosis of cardiovascular diseases. Its software has been designed as a neural network ready to interpret electro-cardiogram signals and to warning of any possible arrhythmia or heart anomaly directly to the user's smartphone or—through IoT—to user's emergency services and/or user's MD in any point of the planet. Cardio Warning=Active Biosensors+Artificial Intelligence+Cardiologic Big Data.

With the data gathered in real time from its wearable biosensors, it performs a continuous monitoring of the heart and the main associated parameters to give a total coverage in the early prevention of heart disease.

The analysis and diagnosis service is the most important part of the system since it accepts not only ECG & apps of our own devices, but it is the basis of a service that interprets any ECG and that is offered to medical centers which do not have any cardiologist, as well as medical insuring companies or patients, field areas first aid units, NGOs, etc., who usually do not have access to perform this kind analysis and diagnosis in any nearby place or at the necessary time.

Through the integration module this service would also be made available to any manufacturer of hardware devices with bio-sensors, who can then add to their devices the ability to obtain the diagnosis of the data sent. In the case of using our own CW device, in addition to having 24-hour monitoring and diagnosis service, it has an emergency warning system which is automatically activated in the case it detects a critical situation that requires it.

The possibility of wearing it as a T-shirt or other costume or attachment allows user to be comfortable wearing it without difficulty, popularizing the solution and making the monitoring and diagnosis of the heart something habitual. It is also included as an option that the system can additionally have a real-time or deferred connection with specialized medical services which are directly accessed with the ECG and the diagnosis.

On the other hand, the health of a person can be monitored from a set of individual medical data that can be extracted, stored, related, and processed. While in the interpretation of such data, health professionals, companies and research institutes can find therapies, conduct studies or adjust measures in the prevention of diseases not only to a user or a typology of specific user but even sometimes to the whole world population.

The blockchain technology features of this invention will allow the collection of “live” health data, where the data collected constantly evolves in relation to the measures that each of the users take in a particular way. Prevention campaigns, application of treatments encouraged by professionals, companies, and health systems will allow not only to taking a “picture of the health” of a person or a group of people, but analyzing the evolution of all of them continuously.

The results of the intervention of many entities with heterodox interests, all them devoted to different areas of health knowledge and with the intensive application of big-data analysis systems, learning algorithms and artificial intelligence will mean a quantitative and qualitative leap in the prevention of diseases and the improvement of people's health.

This facilitates an important technological tool for the global transition towards modern integral healthcare that is more preventive and patient-centered.

The protection of the sovereign identity of the user and the application of blockchain technology of this invention allows access to immutable and constantly updated data so that they cannot be modified or shared without the owner's knowledge. The user as a transmitter of data is the axis of a virtuous circle improving healthcare.

The development of new hardware including wristbands wearables and other small devices wirelessly connected to personal smartphones will allow users to know through contracts with service companies the diagnosis of their health status.

The creation of the user's digital identity will allow management of the value of the data that the user possesses, in addition to limiting access to them both to those who can consult it and to what data can be obtained.

The user will be able to store the data of these analyzes together with other medical information and share them in a distributed and protected network to which the different medical entities can access after due agreement of the value of the user's data.

This modality will not only help preventing the development of some diseases but also will affect the whole health value chain because the easy access to the organized health data of millions of anonymous people will allow us to create new and better treatments, new research, new and better prevention policies.

CW Value chain:
[Data→Information→KnowledgeΔHealth policies]

A platform where the data provided by the user creates an ecosystem in which all participating entities come to solve different existing problems, contributing to each of them an increase in value in their participation in the health ecosystem.

The creation of the user's digital identity and acceptance to share their health data through different devices or existing information creates a virtuous circle that:

It will improve the health of the user through prevention and diagnostics thanks to the access and use of wearables and mobile devices.

It will improve the health of society as a whole thanks to the analysis and learning processes of the data, provided by each of the entities.

Reduce healthcare costs thanks to remote diagnosis and prevention systems, allowing poorly developed healthcare systems to offer high value services at a very low cost.

It will enable health professionals and companies access to information that will allow them to progress in their investigations and develop them in a shorter time.

It will allow managers to develop effective prevention policies at very reduced costs.

Cases of use of services.

CardioWarning.

Cardiovascular diseases account for almost 70% of health expenditure in developed countries and are the greatest cause of human mortality in them.

Periodic, regular and continuous monitoring of the heart would allow an early diagnosis and therefore the application of initial phase solutions that would avoid and/or delay most heart diseases, saving many lives and improving the quality of life of people, in addition to drastically reducing the enormous economic resources involved.

Similarly, the knowledge and/or the medical hardware to perform and especially to analyze electrocardiograms are not accessible to many areas of the world (small healthcare centers, rural areas, remote places with difficult access in less developed countries, etc.), still doing more difficult any early detection of heart disease. The solution proposed by the Cardio-warning software of this invention consists of 5 modules that work as a complete solution for monitoring and early diagnosis of heart disease, including:

ECG analysis and diagnosis system that allows obtaining all the parameters of the electrocardiogram correctly, and performing analysis of the patterns to diagnose such possible heart diseases of the patient.

An inbound/outbound integration module that allows both any type of ECG to be loaded in the system for later analysis and diagnosis, as well as allowing access to patient data to the different associated medical services. This module facilitates access to the service to any person/institution/hardware that needs to perform a diagnosis of an ECG, and allows medical professionals to access the results/ECGs of their patients.

One access point (App, Website) through which user will receive so many ECGs from Cardio-warning hardware devices or from other manufacturers, such as those who want to directly add patients/medical centers. The result will always be the interpretation and possible diagnosis of this ECG.

A hardware device with sensors that allows the monitoring of the patient both of the ECG and of other parameters that allow to expand the diagnosis, such as respiration, temperature and accelerometer. This device will be distributed both with sensors for reading on demand, and in continuous monitoring format (patch, shirt, etc.). The device is directly connected to the access point to obtain an immediate diagnosis and receive the corresponding alerts, as well as an emergency alert system for a serious cardiac situation. A bigdata system that monitors the evolution of ECG and is responsible for finding new previously unknown patterns that evolve to heart disease. This system is the key to even more premature detection of possible cardiovascular problems and future help within medicine and cardiology research. The three major differential factors of the CardioWarning platform of this invention are:

Universal service approach to analysis, interpretation and diagnosis of ECG. Accessible from all over the planet by any hardware device that generates an ECG, any doctor, hospital, health service, institution, corporation that needs it, rural or areas of—difficult access, countries or areas with fewer resources, NGOs, etc.

Designed as a research tool to accelerate the discovery of new patterns and parameters of cardiovascular diagnosis from an EGC database specially classified and exploited by big data technologies.

Global platform for the prevention of cardiovascular health that from a simple and intuitive application and operational in the cloud, will allow users to have a management and control of their cardiovascular health in a continuous way and also make available to their medical service the historical and the real-time information available in an easy and fast way. All this is possible thanks to the integration of:

Analysis, interpretation and diagnosis software based on last generation updated IA technologies, universal service model, Service Agreements to provide service to manufacturers, and devices both on demand and continuous monitoring.

Different sensors that will add to the medical interpretation of the ECG other medical parameters such as heart rate, skin temperature, respiratory rate, fall detection in case of syncopes, blood pressure or control of activity among others. In addition, from the application you can enter subjective parameters, situations, including consumption of medicines, drugs or other substances, etc. that may affect the diagnosis.

Alarm management systems and warnings for important or urgent diagnoses, automatic call in emergency situations (heart attacks, fibrillations, falls or loss of consciousness, . . . ), health recommendations module customized to the diagnosis or specific monitoring and online connection with medical service among other services.

The current model of most health organizations can be optimized by the development and implementation of effective preventive systems, as the Cardio Warning platform.

Once a healthcare organization have the cardiological data of their users and the service offered by Cardio warning integrate to the blockchain platform, it will allow the users through their digital identity by:

Share and sell own cardiological data with the entities of the system in a safe and anonymous manner; ensure the integrity of the data and diagnostic services received; analyze and investigate the problems of cardiological health in specific populations; analyze the efficiency of medicines and prevention policies; reward good practices; reward professional teams; and cost savings.

The CardioWarning platform is based on the collection and analysis of information and the ability to interpret it correctly for the elaboration of an automatic diagnosis. In this way, it allows interpreting an EGC, detecting different types of arrhythmias or making a diagnosis about the ECG or its history (continuous or frequent monitoring), from anywhere in the world.

The CardioWarning platform is based on the collection and analysis of information and the ability to interpret it correctly for the elaboration of an automatic diagnosis. In this way, it allows interpreting an EGC, detecting different types of arrhythmias or making a diagnosis about the ECG or its history (continuous or frequent monitoring), from anywhere in the world.

In this way, any particular user, doctor, hospital, company or institution, will have available an interpretation and diagnostic service of ECGs. In a first phase, we differentiate five models of using this invention:

CardioWarning Service. Back-End Service is aimed at end users, doctors, small hospitals, research centers, health care services, rural or remote areas without means of analysis, NGOs, public health systems in countries with fewer resources, etc. In this case, the strategy involves free access at a particular level for sporadic needs and a quotation for sections as the number of monthly analyzes increases.

CardioWarning Device. Device connected to the CardioWarning platform for analysis and diagnosis that is also capable of performing a quality ECG. Prevention technology that will improve the quality of life of people while providing a better service and reduce costs derived from cardiovascular diseases. Oriented to insurers, hospitals, end users concerned about their health, with risk factors or with a diagnosed cardiovascular disease and in general health systems. Initially there will be two models:

Continuous monitoring device, integrated in a shirt with sensors or attached to the body allowing anyone to obtain a constant ECG reading and the associated diagnosis in their mobile device. This device has a built-in GPS and accelerometer to make emergency alerts in case of serious situations, recommends guidelines or actions based on analysis, diagnosis and context and includes the possibility of incorporating a medical service online or offline for the analysis of diagnosis and possible treatments.

On-demand reading device, indicated for small health centers, doctors not specialized in cardiology, as a complement to medical services displaced abroad, NGOs, and, in short, any health service that needs to make an ECG on time. interpret it and do not have the knowledge or the necessary means to do it.

CardioWarning Services of this invention is also aimed at servicing manufacturers of continuous monitoring devices and manufacturers of wristbands and smart watches that incorporate in the near future the possibility of obtaining an ECG (Apple, Samsung or Fitbit already work on it). The CardioWarning platform of this invention provides in this case the device with the ability to interpret and diagnose the ECG or ECGs as if it were a cardiologist.

The goal of this invention is to popularize the service and to get access to the interpretation and diagnosis of the CardioWarning platform from any ECG reading device. The solution would also be valid for any hospital that has devices that generate the ECG in digital format, since they can obtain the diagnosis from any online access point.

Optionally, Cardio Warning can be delivered as a system itself, comprising all the hardware and software required for providing same results.

Any Health device providing an interface to retrieve health data can be used with Cardio Warning.

Wearable sensors for the customer will vary depending on the kind of heart sensor required (internal, subcutaneous or external) for each patient according to the severity of the disease

CardioWarning Service is a Service platform that responds to any requests for analysis and interpretation of electrocardiograms through IoT or any other monitoring device. It is accessible from any IoT device and can be integrated with any other existing monitoring devices.

The Healthcare Cloud Service Platform of this invention has been designed to be offered for medical data exploitation. The information provided by the devices of this invention, any third-party devices and Cardio Warning Service itself; all them combined provide an outstanding egregious database for health services, laboratories, hospitals or doctors.

CardioWarning Service of this invention include Back-End service for Insurance companies, labs, health-care services, end users, rural services, non-governmental organizations (NGOs) etc. which are remote or without means of analysis. Cardio Warning Service is also focused to work with manufacturers of monitoring devices for improving the automated AI interpretation of electrocardiograms (ECG) and other medical monitoring displays.

The analysis of information helps the research and finally becomes knowledge. This ecosystem provides the healthcare industry with access to different data sources, multiple types of subjects and different pathologies—among many other issues—to prepare their reports, forecasts, studies or future pharmacological developments.

The first service of this invention will be related to cardiology but the ecosystem will be populated by new verticals and services referred to other medical needs, all them related to the healthcare industry.

As the number of users grows and the services in relation to the different pathologies are incorporated and new data can be made available, the platform is revalued because it offers the interveners a unique tool in constant evolution to which there is no better alternative so far.

They can be applied to the data of a service, depending on the service offered, rules of diagnosis, algorithms and machine learning that will learn from the health of users anonymously to prevent irregular behavior.

In addition, the platform enables the possibility of developing other more complex and specific sub-services of this invention, linked to artificial intelligence. Thanks to this system, this possibility will be accessible to more research groups, opening the possibilities to larger group of scientists or academics world-wide.

The system also enables reputation management among professionals in the ecosystem based on different parameters that are approved in the governance of the system or proposed by entities, insurers and healthcare systems to their own medical teams.

The professionals will also be able to value their research, since their publication and access to it will be present/available in the system. The professionals will also be able to value their research, since their publication and access to it will be present/available in the system.

Among the several technical developments of this invention described above there is Blockchain technology, a shared and immutable system of peer-to-peer transactions built by transaction blocks linked cryptographically and stored in a Ledger.

Sophisticated cryptographic techniques enable each participant of the system to interact (issue, save and read information) without establishing prior confidence. The interactions with Ledger are shared among all participants and require verification before processing and storing the information.

The selection of the implementation of this invention on Blockchain technology is determined by the capabilities offered by this protocol to the healthcare sector. Applied to the exchange of information in health services, this Blockchain technology allows to:

Introduce a distributed and secure registry of the exchange of information without having to have a single trusted operator; reduce the costs of services and increase their efficiency thanks to the elimination of intermediaries while processing almost in real time; introduce a set of identifiers into a distributed and secure registry to better protect the patient's identity; share updated patient data almost in real time through a system of records; control access to patient information in a distributed and secure manner; create smart contracts that work in a constant workflow based on rules programmed to access patient data; and provide patients and/or agencies with a sovereign DDI (decentralized digital identity) that will allow them to operate both within the system at first and outside the system once the W3C protocols are applied.

Cardio Warning blockchain platform is a trust network where different actors share and process data and services and which allows not only to record the transactions between the parties but also to quantify the value of each of them according to their typology. This platform would not be possible without the existence of Blockchain technology.

The architecture of the platform consists of two Ledgers, one of which deals with the management of the Digital Identity and another is used to manage the Data Contracts. For scalability needs, the structure of the applications and the experience and development, the platform will be carried out on two frameworks of Hyperledger.

Both Hyperledger frameworks are an initiative of the Linux Foundation, their code is open and hundreds of programmers and companies participate in the development, ensuring its development and continuity. These same frameworks are being sponsored by IBM.

Sudden infant death (or Sudden Infant Death Syndrome—SIDS) is the leading cause of infant death in the first year of life. So far there is no known way to avoid it, but to prevent it.

In Western Europe alone, 2,400 babies die in their cribs every year, and a similar figure in the USA, with o early warning signs. They just stop breathing. During this sudden infant death syndrome (SIDS), the problem arises when these respiratory stops happen after 10 seconds. First, they can cause damage to the nervous system and—in the worst case—the death of the infant. If someone touched the child at that moment, he would simply wake up.

There are in the market some devices to control the baby's breathing and listen the sound and movements made by the baby. The problem is that most of them are not accurate enough and they make too many false alarms that makes them loose reliability and therefore usability.

Cardio Warning system is much more accurate in this SIDS embodiment as its sensitive wireless sensors focus mainly on the heartbeat rate of the infant. along with other features including temperature and movement that have the traditional systems reducing considerably false alarms for numb parents or caregivers. The new sensors are also less cumbersome than the current technology and can be connected to the quilt or directly to the cradle of a baby.

In this way, as soon as an abnormal alteration is confirmed, the sensors alerts by Bluetooth or Wi-Fi to the parent's mobile phone, also generating if required a small discharge to the baby to activate his heart rate again. The developed Cardio Warning algorithm recognizes the heart rate and the movements at logical frequencies and, when it detects a prolonged apnea, it immediately sends a load alarm to the parent's mobile phone. Additionally by using other sensors existing in the market it can resuscitate the babies with a trained stimulus in a proper light way so they recuperate while not get awaken.

The system is designed especially for premature babies and babies who may have some disease, such as a simple cold or several bronchial processes.

The military and/or militarized rescue units such as the Spanish UME (Emergencies Military Unit) uses of this invention include a number of embodiments with modifications to the CW for military use besides the features and means of user data transmission and its protocols as well as the data storage following military security requirements in order to avoid its access by any potential enemy. The CW military version results in a much simpler device than the civil version but with some different functions such as the remote detection of death or life of a soldier to prioritize or not rescue missions, etc. In a military scenario, transmissions will be performed using military standards and devices (i.e. secured radios and protocols).

These real-time biomedical constant sensors can also be used by firefighters and all types of military personnel assistance including determination of the severity of wounds produced. The sensors are automatically synchronized with the node through wireless communications such as Bluetooth, Wi-Fi or radio. All information from the sensors is collected by the node and sent to the cloud by mobile or radio-satellite communications so that it can be displayed to the user in the dashboard of the web application accessible on the website.

These electronic sensors are included in the wearable vest, shirt, patch or other alternative platform of sensors, able to monitor the cardiopulmonary activity and other body parameters. The main sensor normally includes a miniature electronic device designed to control the cardiovascular and respiratory function of the user.

In the Military Cardio Warning version the wearable sensor platform constantly measures and detects the specific vital signs of the user and when any key vital variable seems to be out of range, the application software performs a local pre-alarm analysis first, through low power Bluetooth connection with the smartphone or military equivalent of the user, who communicates with the AI+Big Data center with the encrypted Cloud, which, in turn, if a serious anomaly is confirmed, will activate an alarm and provide the CVD information data together with the user's geolocation—to the command and/or local rescue services, depending on the protocols of the mission.

In the Cardio Warning version for the Sport there are two main different applications studied by Sport Cardiologists specialized in Sport activities.

The first one is referred to the Cardio control of big professional events, such as Cycling events (like the Tour of France), Marathons or Football matches where all athletes are monitored with wearables of size and shape adapted and adjusted to avoid interference of bio-sensor with each specific sport activity, and during the event all participants are monitored from a central Medical station with professional cardiologist ready to stop any of the athletes showing any early symptoms of CVD or any other relevant sport-related detectable parameters.

The second one is devoted to serve average end-user people running or making other amateur sport.

Each specific Cardio Warning wearable equipment is adapted to the sport motion itself specially its wearable sensor platform that constantly measures and detects the vital signs of e user and when any key vital variable seems to be out of range during sporting activity, the application software performs a local pre-alarm. analysis first, through Bluetooth connection with the smartphone of the port user, who in turn communicates with the AI+Big Data center in the Cloud if a detectable anomaly arises.

IN that case, the system will activate an alarm to the user to stop and if no reply is received it will provide the information data together with the user's geolocation—to the emergency services.

### Advantages of Cardio Warnings Are:

Cardio Warning boosts preventive treatment of diseases which can be detected through wearables and portable and/or mobile devices; time reduction for diagnosing and treatment application; reduction of health care system costs as preventive treatment delays or avoids future hospitalization; hospitalization costs are severely reduced as hospitalizations are not required so often due to prevention and due to health specialists perform patient tracking remotely; prevention of a number of deceases in the case of emergency situations when an alarm will raise from the mobile/device in order to send urgent aid message with the GPS detected patient location (internet of things) to the nearest central or local Hearth care unit in case that it is necessary; optimization of resources due to hospitalization reduction; extensible to other pathologies including: Hypertension, Arrhythmias, Congestive Heart Failure, Diabetes, Cancer; improves patient's life quality and autonomy, saving lives in the most critical patients; The best medical specialists can be remotely available, so reducing trips, costs, and providing the best assistance independently of patient location and in real time; and knowledge reutilization and transfer.

CardioWarning includes two ways of helping users for the early prevention of CVD (cardiovascular diseases). Cardio Warning devices are integrated with the CW analysis software of this invention that monitors our heart 24 hours a day and early alerts if any heart problem arise. It also notices by Internet of things (IoT) to emergency services in the event of any critical situation, switching the focus from treating diseases towards tracking first signs of health problems.

Depending of the device retrieving data the way of using Cardio Warning for the final user may vary. Customers using Cardio Warning (health service providers, hospital networks, Health-IT companies, or even independent customers) may use the product as a complete system on their own customized inference rules or as a Cloud service where inference rules can be the ones defined by the customer or connected to main reference Health Care System hospitals.

The way the customer notifies to the final users the results of the process depends on the customer business model and the contract established with the final user.

Cardio Warning software includes an AI model that can identify heart problems and some other health problems by tracking the first symptoms of them through a platform of sensors (some external wearables and others in the form of applications for Android-based smartphones, iPhone or other) that allows specialists to perform medical and biometric actions in which it is necessary to monitor the patient and transfer the data remotely.

Cardio Warning system works with a suitable wearable with wide functionality with built-in sensors with electrodes and non-invasive electronic technology to perform biometric readings of the user with such small adhesive electrodes allows to compile in real time the biometric data of the user, which are sent to his mobile phone. Biometric data gathered from these sensors is sent to the smartphone of the user by Bluetooth for the preliminary analysis with this Cardio Warning smartphone APP to determine cardiopulmonary activity by means of a miniature electronic device, designed to control the cardiovascular and respiratory function of the user. After that the data is transferred to the Cloud for deep analysis. In the case of cardio dysfunction, the user gets a notification with recommendations of how to prevent unwanted consequences. If the user does not react to the alarm message, it is redirected with the latest GPS location of the user to his MD, Clinical center, user's relatives and/or emergency services phone numbers previously established.

The Healthcare Cloud Service Platform of this invention can also be offered to third parties worldwide for telemedicine data exploitation in favor of its patients with further recommendations.

Specific versions of CardioWarning of this invention can be made available to be prescript by doctors as an aid in the prevention and the early diagnosis of heart disease and/or some other determined diseases. With different kinds of Wearable Biomedical technology sensors available or to be available in the market or specifically developed for CardioWarning, and developing solutions for the prevention of different diseases including Diabetes, Hypertension, Cancer, as long as biosensors evolve.

The procedure and model where if the user does not react to the alarm message, this message is redirected to the telephone numbers of his doctor, medical center or other alternative previously established, and even the emergency telephone number of the country and the locality in which there may be Risk of severe CVD.

The system where the components for accurate disease prevention are the wearable platform of High Resolution Biosensors that receives and emits by Bluetooth or Wi-Fi the signals to the smartphone and then to the Cloud storage for further detection of arrhythmias and/or other disorders such as respiratory diseases. It captures the main vital signs such as: EKG/ECG and heart rate, respiratory rate and skin temperature.

The procedure and business model where Cardio Warning Service and Systems are used for detecting sudden infant death.

The procedure and model where Cardio Warning Service and Systems are used for detecting heart beat status for prioritizing rescue missions in both civil and military missions. The Interfaces and protocols used for connecting Cardio Warning Service and Systems to civil and military communication devices (used by rescue teams. The procedure and model where Universal service approach to analysis, interpretation and diagnosis of ECG. Accessible from all over the planet by any hardware device that generates an ECG, any doctor, hospital, health service, institution, corporation that needs it, including rural areas of difficult geographical access, countries or areas with fewer resources, NGOs, etc. The procedure and model where Cardio Warning has been designed as a research tool to accelerate the discovery of new patterns and parameters of cardiovascular diagnosis from a database of EGCs specially classified and exploited by Big Data technologies. The procedure and model where Cardio Warning is designed as a Global platform for the prevention of cardiovascular health from a simple and intuitive application operational in the Cloud, that will allow users the outlook and management of their cardiovascular health in a continuous way and also make available their medical record both the historical and the real-time information. All that made available in an easy and fast way.

The Analysis, interpretation and diagnosis software based on the latest IA technologies, Universal service model, Service Agreements to provide service to manufacturers, Device on demand and Continuous monitoring device. This universal and compatibility approach will allow access to a growing up-to-date database, which will allow continuous training and refinement of detection algorithms.

The incorporation of different bio-sensors to measure other medical parameters such as heart rate, skin temperature, respiratory rate, blood pressure or control of activity among others will be added to the medical interpretation of the ECG. And from the application it will be possible to introduce subjective parameters, situations, consumption of medicines or other substances, etc. that may affect the diagnosis.

The management of alarms and warnings for important or urgent diagnoses, automatic call in emergency situations (heart attacks, fibrillations, syncope-related falls or loss of consciousness, etc.), health recommendations module, customized to the diagnosis or specific monitoring and online connection with medical service among other services.

The procedure and model where The payment for access to anonymous databases by researchers and pharmacists will allow to offer diagnostic services at a very low cost and affordable for all sectors of the population.

The procedure and model where the use of blockchain technology will allow security and privacy unattainable by traditional centralized systems.

The CardioWarning Service. Back-End Service, which is aimed at end users, doctors, small hospitals, research centers, health care services, rural or remote areas without means of analysis, NGOs, public health systems in countries with fewer resources, etc. In this case it should involve free access at least at a particular level for sporadic needs and a quotation for sections as the number of monthly analyzes increases.

The procedure and model where CardioWarning Device connected to the CardioWarning platform for analysis and diagnosis that is also capable of performing good quality ECGs.

The procedure and model where providing a CW specialized service oriented to medical insurers, hospitals, end users concerned about health, with risk factors or with a diagnosed cardiovascular disease and in general all health systems/organizations. The Continuous Monitoring Device, integrated with a T-shirt or other article of clothing with bio-sensors or in a wearable patch to be attached to the body for tracking the main vitals. It allows anyone to obtain a constant ECG quality reading and the associated quality early diagnosis displayed in the client's smartphone. This device has a built-in GPS and accelerometer to make IoT emergency alerts/calls in case of syncope or any other serious situations, recommends guidelines or actions based on analysis, diagnosis and context and includes the possibility of incorporating a medical service online or offline for the analysis of the diagnosis and possible treatments.

The procedure and model where On-demand reading device, indicated for small health centers, doctors not specialized in cardiology, as a complement to medical services displaced abroad, NGOs, and, in short, any health service that needs to make an ECG on time, interpret it and do not have the knowledge or the necessary means to do it.

The procedure and model where CardioWarning Service is aimed at manufacturers of continuous monitoring devices (in development) and manufacturers of wrists and smart watches that incorporate in the near future the possibility of obtaining an ECG (Apple, Samsung or Fitbit already work on it). The CardioWarning platform in this case proposes the ability to interpret and diagnose the ECG or ECGs as if it were a cardiologist. The objective is to popularize the service and to get access to the interpretation and diagnosis of the CardioWarning platform from any ECG reading device. The solution would also be valid for any hospital that has devices that generate the ECG in digital format, since through an online access point they can obtain the diagnosis.

The procedure and model where In a second phase and when a sufficiently large database of ECGs is available, another model will be implemented with two approaches: 1. The use of this huge Database of classified electrocardiograms owned by GI biomed that can be used for research by laboratories, universities and research centers with the aim of improving treatments and the prevention of cardiovascular diseases. 2. Exploitation of this database using Big data techniques to locate patterns that are not yet known and that are likely to develop heart disease in the future. These two lines would allow Cardio Warning System to advance enormously in the prevention of cardiovascular diseases from a very early stage of the same, saving as many lives as possible, which is the first priority for all of us and with an expected huge cost-savings for the existing health organizations.

Cardio Warning Sovereign Digital Identity. The creation of the sovereign digital identity-SDI will allow the user to manage the value of their data and may limit access to them or establish what data to share. This innovation affects the entire health value chain. Access to the health data of millions of anonymous people will allow creating new and better treatments, while new ad-hoc research and better prevention policies.

Cardio Warning Services from the mobile phones. Devices that connects to our cell phones, monitors our health and brings us closer to analysis and diagnosis services while dramatically reducing health costs. Not only in developed countries, but all around the world, millions of people will be able to analyze their health, heart, diabetes, DNA analysis data from the smartphone preserving their anonymity, the future of the prevention in health.

The procedure and model in the Cross connection where the health of a person can be represented in a set of data that has been extracted, stored, related, and processed. In the interpretation and analysis of them, health professionals, research institutes, or public healthcare organizations can find therapies, studies or adjust measures in the prevention of diseases not only to some user or a typology of specific users but to groups even as large as the whole world population.

The procedure and model where Universal service approach to analysis, interpretation and diagnosis of ECG is included. Accessible from all over the planet by any hardware device that generates an ECG, any doctor, hospital, health service, institution, corporation that needs it, remote areas with difficult geographical access, countries or areas with fewer resources, NGOs, etc.

The procedure and model where the CW Research tool is included. A tool to accelerate the discovery of new patterns and parameters of cardiovascular diagnosis from an EGC database specially classified and exploited by Big Data technologies.

The procedure and model where Global Platform for the prevention of cardiovascular health is included. A simple, intuitive and operative application in the Cloud that will allow CW users to have a vision and management of their cardiovascular health in a continuous way and also to make available to the medical service the personal cardiovascular related history and the Real-time relevant information in an easy and fast way.

1. An automated system with its associated devices that includes wearable sensors and mobile apps for autonomous patient monitoring, diagnosis and alert device and its related software including the inference rules, Artificial Intelligence, and Big Data algorithms and techniques for the prevention of diseases. The apparatus used for detection and measurement of health signals in order to remotely detect in advance any disease symptom that can be treated preventively in order to avoid or delay more aggressive treatment or even hospitalizations.

2. The system of claim 1, including an interface to obtain data from remote devices, customers, and institutions. Cardio Warning receives medical data from sensors located at patients with diseases and from http and REST compatible applications. All the information received is stored in a knowledge database generated with support of reference centers (cardiology, diabetes, cardiovascular, etc.) and it is processed by using inference procedures, providing results and reports to the healthcare professionals who is attending, diagnosing, and remotely tracking in real time to patients, preventing the evolution of the disease.

3. The system of claim 1, including a procedure to transfer information received from remote devices and internet connections to a Cloud storage. It includes the complete software for remote patient monitoring, detection of disease patterns, anticipation of the disease evolution through inference rules and massive data analytics techniques, and the alert system for health care professionals, insurances, and any third party receiving notifications of diagnoses.

4. The system of claim 1, including a procedure of data analyzing in order to detect anomalies susceptible to correspond to a particular disease generating a report and/or an alert in case it is necessary.

5. The system of claim 1, including patterns and algorithm parameters used for detection of diseases (i.e.: arrhythmias) and also algorithms for preventive detection of diseases through data retrieval from wearables and/or internet connections, and the post analysis via big data techniques.

6. The system of claim 1, including knowledge database of patients' data remotely acquired. Diagnosis Aid Service for diseases through the remote patient monitoring anticipating the disease evolution through inference rules and massive data analytics techniques.

7. The system of claim 1, including a procedure for tracking remotely patients through remote data acquisition, storage of received data in cloud resources, and analysis of stored data using knowledge database for detecting symptoms of diseases creating appropriate alerts for patients.

8. A method for prevention of a number of deceases in the case of emergency situations when an alarm will raise from the mobile/device in order to send urgent aid message with the GPS detected patient location (internet of things) to the nearest central or local Hearth care unit in case that it is necessary.

9. The method of claim 8, where wearable sensors for the customer will vary depending on the kind of sensor required (internal, subcutaneous, external, or a web protocol) for each patient according to the severity of the disease.

10. The method of claim 8, where the information received is stored in a Disease Knowledge Database generated with the support of the reference centers (Cardiology, Cardiovascular, Diabetes, etc.) and it is processed using inference procedures looking for patterns defined by the reference centers in order to early (prematurely) detect any sign of disease providing results and reports to the local healthcare professionals who are attending, diagnosing, and remotely tracking patients in real time.

11. The method of claim 8, where this system is extensible to other pathologies including Hypertension, Arrhythmias, Congestive Heart Failure, Diabetes, Cancer, and many other medical conditions/diseases, etc.

12. A Cardio Warning system wherein health service providers, hospital networks, Health-IT companies, or even independent customers may use the product as a complete system on their own customized inference rules or as a Cloud service where inference rules can be the ones defined by the customer or connected to main reference Health Care System hospitals.

13. The system of claim 12, including a data warehouse created (content and structure) from data retrieved through CardioWarning service and the exploitations mechanisms.

14. The system of claim 12, used to expose close and accessible devices that may help customers to overpass critical situations (i.e.: closest defibrillators) to CardioWarning service customers.

15. The system of claim 12, used to register and update the status and localization of the devices described in the previous paragraph.

16. The system of claim 12, including defining areas where there are devices located and with their usability status known in order to be employed in critical situations (i.e.: defibrillators).

17. The system of claim 12, including a specialized health monitoring software and diverse alternatives of hardware consisting in smart wearable devices of heart surveillance by key biosensors included in patches, t-shirts or vests, all synchronized with the Apps of this invention specifically designed for a variety of smartphones.

18. The system of claim 12, including a platform of sensors that may optionally include several other additional devices that continuously follows other vital signs including pulse, oxygen in blood, airflow (respiration), body temperature, electrocardiogram, glucose meter, galvanic skin response (sweating), blood pressure and patient position (accelerometer).

19. The system of claim 12, where the information generated is locally encrypted and processed in the Cloud, using Big data and Internet of Things technologies. Cardio Warning cardiac software includes the algorithm that looks at any anomalies in heart function and provides recommendations for taking preventive measures.

20. The system of claim 12, wherein, in the case of cardio dysfunction, the user receives an automatic notification (based on artificial intelligence that includes in-depth analysis to detect serious cardiac abnormalities) with recommendations on how to prevent unwanted consequences, including the nearest defibrillator to his GPS location.
